(19) United States
(12) Patent Application Publication
(10) Pub. No.: US 2014/0112898 A1
(43) Pub. Date: Apr. 24, 2014

(54) UNIQUE POPULATION OF REGULATORY T CELLS THAT REGULATE TISSUE REGENERATION AND WOUND HEALING

(71) Applicants: Diane Mathis, Brookline, MA (US); Christophe Benoist, Brookline, MA (US); Dalia Burzyn, Cambridge, MA (US); Amy Wagers, Brookline, MA (US)

(72) Inventors: Diane Mathis, Brookline, MA (US); Christophe Benoist, Brookline, MA (US); Dalia Burzyn, Cambridge, MA (US); Amy Wagers, Brookline, MA (US)

(73) Assignee: PRESIDENT AND FELLOWS OF HARVARD COLLEGE, Cambridge, MA (US)

(21) Appl. No.: 14/008,955

(22) PCT Filed: Apr. 2, 2012
(86) PCT No.: PCT/US12/31874

§ 371 Date: Jan. 8, 2014

Related U.S. Application Data
(60) Provisional application No. 61/470,205, filed on Mar. 31, 2011.
(60) Provisional application No. 61/470,959, filed on Apr. 1, 2011.

(51) Int. Cl. A61K 35/14; A61K 45/06

(52) U.S. Cl. CPC A61K 35/17; A61K 45/06
USPC 424/93.71; 435/325; 435/375; 435/377

(57) ABSTRACT

A unique type of regulatory T cell has been identified in muscle. These tissue-regenerative Treg cells play a role in regulating wound healing. These cells, as well as agents that control their differentiation and/or activity and agents produced by the cells, can be used to modulate wound healing and the differentiation of muscle cells.

### GOVERNMENT FUNDING

This invention was made with Government support under National Institutes of Health award R01 AI051530. The Government has certain rights in the invention.

### BACKGROUND

Wound healing is a fundamental biological process that must operate efficiently and over the course of a lifetime to ensure survival of the organism. It proceeds in two major stages. The first of these involves recruitment, activation, and expansion of inflammatory cells at the wound site. The second involves waning of the inflammation in concert with mobilization of tissue-repair processes. The identification of cell populations involved in these processes and factors that control their differentiation and activity as well as downstream factors produced by such cell populations would be of great value in modulating wound healing processes.

### SUMMARY

As described herein, a unique population of Foxp3⁺CD4⁺ T regulatory cells (Tregs) has been discovered in muscle tissue. These cells infiltrate injured muscle as the inflammatory stage transitions to the regenerative stage. These tissue-regenerative Treg cells are required for efficient muscle repair, and their numbers fluctuate in muscle diseases such as muscular dystrophy, and with aging (which is accompanied by less effective wound healing).

In addition to their tissue-regenerative properties, according to a number of criteria, these Treg cells have a unique phenotype, distinct from that of previously described regulatory T cell populations. These Treg cells express high levels of the following molecules: IL10, Pcsk1, Areg, Pcyt1a, Frmd5, Ccr1, Ccr3, Lyn, Arnt2, Pparg, Ctsh, Havcr2(TIM3), Gpr55, Il23r, Itgae, Ccr6, Dgat2, Rorc, CD74, Il1r2, Il1r11, CD200r1 and Trf (or IL10, Pcsk1, Areg, Ccr1, Arnt2, Pparg, Npnt, Itgae, Ccr6, Havcr2(TIM3), Gpr55, Il23r). In one aspect, the invention pertains to compositions of Foxp3+CD4+ regulatory T (Treg) cells isolated from muscle, which Treg cells exhibit tissue regenerative properties.

In one embodiment, the Treg cells are characterized by transcription of IL10, Pcsk1, Areg, Pcyt1a, Frmd5, Ccr1, Ccr3, Lyn, Arnt2, Pparg, Ctsh, Havcr2(TIM3), Gpr55, Il23r, Itgae, Ccr6, Dgat2, Rorc, CD74, Il1r2, Il1r11 (ST2), CD200r1 and Trf (or of IL10, Pcsk1, Areg, Ccr1, Arnt2, Pparg, Npnt, Itgae, Ccr6, Havcr2(TIM3), Gpr55, Il23r) at levels higher than splenic or lymph node Treg cells.

In yet another embodiment, the invention pertains to a method of administering comprising administering a composition of the invention to a subject.

In one embodiment, the composition is administered systemically. In one embodiment, the composition is administered directly to muscle tissue. In one embodiment, the composition is administered directly to a wound. In one embodiment, the composition is administered to a subject having an injury to muscle tissue.

In one embodiment, the composition is administered to the subject at the time of injury. In one embodiment, the composition is administered to the subject several days after the injury.

In one embodiment, the subject has a degenerative muscle condition. In one embodiment, the subject is of advanced age.

In one embodiment, the subject has diabetes.

In one embodiment, a method of the invention further comprises administering an anti-inflammatory agent.

In one embodiment, the invention pertains to a method of promoting wound healing comprising contacting muscle cells with a composition of the invention. In one embodiment, the step of contacting occurs in vivo.

In another aspect, the invention pertains to a method of producing a population of cells enriched for tissue regenerative Treg cells, the method comprising obtaining a starting population of cells comprising Treg cells and selecting or inducing cells from the starting population that express IL10, Pcsk1, Areg, Pcyt1a, Frmd5, Ccr1, Ccr3, Lyn, Arnt2, Pparg, Ctsh, Havcr2(TIM3), Gpr55 and Il23r (or IL10, Pcsk1, Areg, Ccr1, Arnt2, Pparg, Npnt, Itgae, Ccr6, Havcr2(TIM3), Gpr55, Il23r) at levels higher than the bulk populations of splenic or lymph node circulating Treg cells, to thereby produce a population of cells enriched for tissue regenerative Treg cells.

In one embodiment, the method further comprises culturing the cells ex vivo in order to expand them.

In one embodiment, the cells are cultured in the presence of at least one agent selected from the group consisting of: at least one cytokine, muscle cell extract, and at least one myokine.

In one embodiment, the starting population of cells comprises cells derived from muscle.

In one embodiment, the invention pertains to a composition produced by a method of the invention.

In yet another embodiment, a method of the invention further comprises administering the cells to a subject.

In one embodiment, a method further comprises contacting the population of cells enriched for tissue regenerative Tregs with muscle cells or muscle cell progenitors. In one embodiment, the step of contacting occurs in vivo.

In another aspect, the invention pertains to a method of promoting wound healing comprising contacting a wound of a subject in need of wound healing with at least one agent that promotes the development of Treg cells.

In one embodiment, the at least one agent is selected from the group consisting of: anti-CD3, at least one PPARγ agonist, at least one thiazolidinedione-like drug, and IL-2/anti-IL-2 complexes.

In one embodiment, the at least one agent is pioglitazone or another PPARγ agonist.

In one aspect, the invention pertains to a method of promoting wound healing comprising contacting a wound of a subject in need of wound healing with an agent derived from tissue-regenerative Treg cells.

In one embodiment, the agent is selected from the group consisting of: IL-10, Areg (amphiregulin), Havcr2 (TIM3), and Npnt (nephronectin).

In one embodiment, the agent is selected from the group consisting of: IL-10 and Havcr2 (TIM3).

In yet another aspect, the invention pertains to a method of promoting muscle cell development ex vivo, comprising contacting cells capable of developing into muscle cells with the composition of the invention.

In one embodiment, a method of the invention further comprises contacting the cells capable of developing into muscle cells with macrophages or at least one agent produced by macrophages.

In one embodiment, a method of the invention further comprises contacting the cells capable of developing into muscle cells with at least one agent selected from the group consisting of: at least one cytokine, muscle cell extract, and at least one myokine.

In yet another aspect, the invention provides use of a composition comprising tissue-regenerative Treg cells in the manufacture of a medicament for therapy, such as the treatment of a disease or disorder. In one embodiment, the invention provides use of a composition comprising tissue-regenerative Treg cells in the manufacture of a medicament for promoting wound healing. In one embodiment, the invention provides use of a composition comprising tissue-regenerative Treg cells in the manufacture of a medicament for treatment of a degenerative muscle condition. Moreover, other uses of the compositions of the invention in therapy are described herein.

Other features and advantages of the invention will be apparent from the following detailed description and figures, and from the claims.

### DESCRIPTION OF DRAWINGS

FIGS. 1A-1B show expansion of the tissue-regenerative Treg population at the site of muscle injury as inflammation is waning and regeneration is beginning. 8-wk-old C57BL/6 mice were injured i.m. with cardiotoxin (30 μg/ml) and the muscle infiltrate was analyzed after 1, 4, 8 and 16 days by flow cytometry. Each sample was stained for analysis of Tregs (FIG. 1A) and myeloid cells (FIG. 1B).

FIGS. 2A-2E show that ablating tissue-regenerative Tregs inhibits muscle regeneration after wounding. 8-wk-old C57BL/6 Foxp3-DTR⁺ mice or their Foxp3-DTR-littermates were treated with diptheria toxin (DT) to specifically deplete Tregs and then injured i.m. with cardiotoxin (30 μg/ml). After 8 days, the muscle infiltrate was analyzed by flow cytometry (FIG. 2A), and muscle regeneration was analyzed by hematoxylin and eosin H&E staining (FIG. 2B). When injury and repair occur in the absence of tissue regenerative Tregs the initial inflammation is not resolved, as shown by the accumulation of CD11b⁺Ly6c high monocytes (FIG. 2A) and persistence of a mononuclear infiltrate (FIG. 2B, right panel). In addition, in the absence of Tregs muscle regeneration is impaired, as indicated by the reduced numbers of centrally nucleated (newly regenerated) myofibers in the injured area (FIG. 2B), by increased fibrosis levels (FIG. 2C), by alterations in the transcriptional profile of muscle regeneration (FIG. 2D) and by reduced clonal efficiency of skeletal muscle progenitors (FIG. 2E).

FIGS. 3A-3C show that tissue-regenerative Treg cells are unique. A comparative gene-expression analysis of Tregs from spleen and from injured skeletal muscle shows that more than 500 genes are differentially expressed (up- or down-regulated) between these two Treg populations (FIG. 3A). According to Principal Component Analysis (PCA) the muscle Tregs, are clearly different from Tregs isolated from all lymphoid tissues, and most closely resemble Tregs resident in fat. The gene profiles of muscle Tregs and muscle T conventional CD4+ cells are distinct while still remain distinguishable from this population (FIG. 3B). The gene expression profiles of muscle Tregs and muscle conventional CD4+ T cells are also distinct (FIG. 3C).

FIG. 4 shows an expanded population of tissue-regenerative Tregs in murine models of muscular dystrophy. The infiltrate of diaphragms and hind limb muscles from 4-wk-old mdx (Murine X-linked muscular dystrophy) or control (C57BL/B10) mice were analyzed by flow cytometry.

FIGS. 5A-5B show a reduced population of tissue-regenerative Tregs in aged mice. The increase in muscle Treg frequency after cardiotoxin injury is not observed in 30-wk-old mice (FIG. 5A). The low Treg frequency in older mice correlates with an increased accumulation of total T cells in the injured muscle (FIG. 5B).

FIGS. 6A-6C show that tissue-regenerative Treg cells are unique. 8 week old C57BL/6-Foxp3-IRES-GFP mice were injured intramuscularly (i.m.) with cardiotoxin (30 μg/ml) and after 4 days Tregs from the muscle and spleen were single cell-sorted by flow cytometry. After PCR amplification, the CDR3 region of the TCRa and TCRb chains were sequenced and analyzed using IMGT/V-QUEST. FIG. 6A illustrates that a significant proportion of Tregs isolated from injured muscle are clonally expanded. FIG. 6B is a summary bar graph illustrating the frequency of clonal muscle Tregs in 3 different mice. FIG. 6C shows the muscle Treg sequences for TCRa and TCRb that were found in different individual mice in independent experiments, suggesting the Tregs are responding to a particular antigen in the muscle.

FIGS. 7A-7D show that modulation of Treg frequency affects muscle damage in dystrophic mice. FIG. 7A shows the results of flow cytometry assays demonstrating that the frequency of muscle Tregs in dystrophic Mdx mice is augmented by treatment with IL2/anti-IL2 complexes. FIG. 7B shows the results of a creatine kinase assay showing that the increase in Tregs in mice treated with IL2/anti-IL2 complex correlates with a significant reduction in the levels of serum creatine kinase, a marker of muscle damage. FIG. 7C shows the results of flow cytometry assays demonstrating that treatment of Mdx mice with anti-CD25 decreases the frequency of Tregs in the spleen but not in the muscle, although it affects CD25 expression in both tissues. FIG. 7D shows the results of a creatine kinase assay showing that anti-CD25 treatment correlates with increased muscle damage, as measured by increased levels of serum creatine kinase.

### DETAILED DESCRIPTION

The present invention is based, at least in part, on the discovery of a unique population of regulatory (Treg) T cells in muscle tissues. Treg cells are a lineage of CD4+ T lymphocytes specialized in controlling autoimmunity, allergy and infection (Sakaguchi, S. et al. Immunol Rev. 212, 8-27 (2006); Fontenot and Rudensky, Nat. Immunol 6, 331-337 (2005)). Initially characterized by surface-display of the interleukin (IL)-2 receptor α chain, CD25, and later by expression of the transcription factor Foxp3, naturally occurring Treg cells normally constitute about 10-20% of the CD4+ T lymphocyte compartment. Typically, they regulate the activities of T cell populations, but they can also influence certain innate immune system cell types (Maloy et al., J. Exp. Med. 197:111-119 (2003); Murphy et al., J. Immunol. 174:2957-2963 (2005); Nguyen et al., Arthritis Rheum. 56, 509-520 (2007)).

As described herein, a population of unique-tissue regenerative Tregs has been identified in muscle. These cells play a critical role in regulating wound healing. Therefore, these cells, as well as agents that control their differentiation and/or activity and agents produced by the cells, can be used to modulate wound healing and the development of muscle cells.

Tissue regenerative Treg cells are characterized by the expression of a unique set of genes. We identified a constellation of gene transcripts, which, as an ensemble, represent a muscle-Treg-specific gene expression signature. Certain of them (IL10, Pcsk1, Areg, Pcyt1a, Frmd5, Ccr1, Ccr3, Lyn, Arnt2, Pparg, Ctsh, Il1r11 (ST2), CD200r1) are expressed highly preferentially in muscle and fat Tregs vis a vis Tregs from other sites as well as conventional T cells from anywhere. Others (Itgae, Ccr6, Dgat2, Rorc, CD74, Il1r2, Trf, Ifit1, Pdgfb) are well expressed in muscle Tregs, and at lower levels in immune tissue Tregs, but are not expressed in fat Tregs. A few genes (Havcr2(TIM3), Gpr55, Il23r) are highly preferentially expressed in muscle Tregs vis a vis all other T cell types examined to date. The compositions and methods described herein take advantage of the properties of these cells by providing, inter alia, compositions comprising these cells. In addition, the invention provides methods by which populations of these cells can be made, methods by which the cells and/or products that regulate these cells and/or products that are made by these cells can be used (e.g., in vivo or ex vivo). In addition, the invention provides methods of identifying agents which modulate the differentiation and/or activity of these cells.

### I. DEFINITIONS

So that the invention may be more readily understood, certain terms are first defined.

As used herein, the term “regulatory T cell” (or “T regulatory cell” or “Treg”) refers to a CD4⁺CD25⁺Foxp3⁺ T cell that negatively regulates the activation of other T cells, including effector T cells, as well as innate immune system cells. Treg cells are characterized by sustained suppression of effector T cell responses. Traditional or conventional Treg cells can be found, e.g., in the spleen or the lymph node or in the circulation.

As used herein, the term “tissue-regenerative Treg” cell refers to a CD4⁺CD25⁺Foxp3⁺ Treg cell that has tissue-regenerative properties. These cells can be found in injured muscle tissue. Tissue-regenerative Treg cells produce a different profile of cytokines than spleen or lymph node Treg cells and muscle and other T conventional cells. For example, Treg from muscle express at least 3 fold more of each of the following genes than Treg isolated from spleen: IL10, Pcsk1, Areg, Ccr1, Arnt2, Pparg, Npnt, Itgae, Ccr6, Havcr2(TIM3), Gpr55, Il23r. As used herein, the term “myokine” refers to peptides or polypeptides derived from muscle cells.

As used herein, the term “muscle cells” refers to those cells making up contractile tissue of animals. Muscle cells are derived from the mesodermal layer of embryonic germ cells. Muscle cells contain contractile filaments that move past each other and change the size of the cell. They are classified as skeletal, cardiac, or smooth muscles.

As used herein, the term “cells that can differentiate into muscle cells” refers to stem cells and muscle progenitor cells that can differentiate into muscle cells.

As used herein, the term “an agent that promotes the differentiation and/or proliferation of Treg cells” refers to one or more agents that cause existing Treg cells to proliferate or which favor the differentiation of Treg cells from cells capable of differentiating into Treg cells (e.g., naïve T cells).

As used herein, the term “PPARγ agonist” refers to an agent that serves as an agonist of a peroxisome proliferator-activated receptor (e.g., PPARγ). Exemplary such agonists include Thiazolidinedione-like drugs or TZDs which act by binding to PPARγ. Exemplary such agents include Rosiglitazone (Avandia), Pioglitazone (Actos), and Troglitazone (Rezulin), Galida (tesaglitazar), and Aleglitazar. Other agents include MCC-555, rivoglitazone, and ciglitazone.

As used herein, the term “IL-2/anti-IL-2 complexes” refers to complexes of IL-2 with anti-IL-2 antibody.

As used herein, the term “recipient” refers to a subject into whom a cell, tissue, or organ graft is to be transplanted, is being transplanted, or has been transplanted. The term “syngeneic” refers to situations in which the donor and the recipient are the same individual. An “allogeneic” cell is obtained from a different individual of the same species as the recipient and expresses “alloantigens,” which differ from antigens expressed by cells of the recipient. A “xenogeneic” cell is obtained from a different species from that of the recipient and expresses “xenoantigens,” which differ from antigens expressed by cells of the recipient.

A “donor” is a subject from whom a cell, tissue, or organ graft has been, is being, or will be taken. “Donor antigens” are antigens expressed by the stem cells, tissue, or organ graft to be transplanted into the recipient.

Unless otherwise defined, all technical and scientific terms used herein have the same meaning as commonly understood by one of ordinary skill in the art to which this invention belongs. All publications, patent applications, patents, sequences, database entries, and other references mentioned herein are incorporated by reference in their entirety.

### II. COMPOSITIONS COMPRISING TISSUE-REGENERATIVE TREG CELLS

In one embodiment, a population of cells enriched for tissue-regenerative cells can be obtained from a starting population of cells that includes a smaller number of tissue-regenerative cells which are then expanded to produce a population of cells enriched for tissue-regenerative Treg cells.

In another embodiment, a population comprising traditional Treg cells (e.g., isolated from spleen or lymph node or from the circulation) that does not comprise detectable levels of tissue regenerative Treg cells can be cultured to obtain tissue-regenerative Treg cells.

In one embodiment, a starting population of cells is contacted with one or more factors that promote the growth and/or differentiation of Treg cells. For example, exemplary factors include one or more cytokines, e.g., IL-10 or TGFβ. Other exemplary factors include one or more chemokines. Such factors can be obtained from a commercial source, or can be produced using standard protein production and purification methods, e.g., by expression in a cultured cell system and affinity purified. In another embodiment, the cells can be cocultured with cells expressing one or more cytokines or chemokines or may be genetically engineered to express such molecules using standard protocols.

In one embodiment, the starting population of cells is contacted with one or more peroxisome proliferator-activated receptor (PPARγ) agonists. Exemplary such agonists include Thiazolidinedione-like drugs or TZDs which act by binding to PPARγ. Exemplary such agents include Rosiglitazone (Avandia), Pioglitazone (Actos), and Troglitazone (Rezulin), Galida (tesaglitazar), and Aleglitazar. Other agents include MCC-555, a powerful antidiabetic agent, rivoglitazone, and ciglitazone.

In another embodiment, a starting population of cells is contacted with at least one thiazolidinedione-like drug. In another embodiment, a starting population of cells is contacted with pioglitazone.

In one embodiment, a starting population of cells is contacted with IL-2/anti-IL-2 complexes or a genetically engineered version of IL-2. Such IL-2:anti-IL-2 monoclonal antibody (mAb) complexes have been shown to promote selective Treg proliferation (Boyman et al., Expert Opin Biol Ther. 2006 December; 6(12):1323-31). IL-2 can be genetically modified to regulate target cell specificity (Levin, A. M. et al. Nature, Mar. 25, 2012, epub: doi: 10.1038/nature10975).

In one embodiment, a starting population of cells is contacted with anti-CD3 antibodies. Anti-CD3 treatment has been shown to promote selective Treg proliferation (Nishio et al., J. Exp. Med. 2010 Aug. 30; 207(9):1879-89).

In another embodiment, a starting population of cells can be cocultured with cells, e.g., muscle cells and/or macrophages in order to promote the proliferation and/or differentiation of tissue-regenerative Treg cells. In another embodiment, a starting population of cells can be cultured with supernatants or purified factors derived from such cells. In another embodiment, a cell or tissue extract can be added to cultures (e.g., a muscle cell or muscle tissue extract). In yet another embodiment, one or more purified myokines or cells expressing myokines can be added to cultures.

In another embodiment, one or more factors made by tissue-regenerative Treg cells can be used to augment differentiation or proliferation of such cells (e.g., in an autocrine fashion). Exemplary such factors include: IL-10, Areg (amphiregulin), Havcr2 (TIM3) and Npnt (nephronectin) (or soluble derivatives thereof, e.g., fusion proteins) or molecules which bind thereto.

A starting population of cells can be cultured (e.g., in the presence of one or more of the agents and/or cell types described herein) until the population of tissue-regenerative Treg cells reaches a certain level (e.g., about 30% of the population, about 40% of the population, about 50% of the population, about 60% of the population, about 70% of the population, about 80% of the population, about 90% of the population, about 1000% of the population) to thereby obtain a population which has been enriched for tissue-regenerative Treg cells. In one embodiment, the cells are used as an enriched population that comprises non-Treg cells. In another embodiment, the tissue-regenerative Treg cells may be purified from any non-tissue-regenerative Treg cells in the enriched population, e.g., based on their expression of CD4, Foxp3, and/or PPARγ.

As demonstrated in the Examples, flow cytometry can be used to purify Treg cells (e.g., to purify Tregs from muscle tissue, for example using the cell infiltrate from injured muscle tissue). Tregs can be isolated using flow cytometry by, for example, gating on CD3+CD4+ cells and sorting for cells that are CD25^{high} and CD127^{low}. Tissue regenerative Treg cells can then be identified and isolated, as compared with non-tissue regenerative Treg cells, based on their unique gene expression profile, as described further below.

The identity of the cells as tissue regenerative Treg cells may be confirmed, e.g., using gene expression methods. For example, in one embodiment, the expression profile of the cells can be tested and cells that express at least one of: IL10, Pcsk1, Areg, Pcyt1a, Frmd5, Ccr1, Ccr3, Lyn, Arnt2, Pparg, Ctsh, Havcr2(TIM3), Gpr55, Il23r, Itgae, Ccr6, Dgat2, Rorc, CD74, Il1r2, Il1r11 (ST2), CD200r1 and Trf or at least one of IL10, Pcsk1, Areg, Ccr1, Arnt2, Pparg, Npnt, Itgae, Ccr6, Havcr2(TIM3), Gpr55 and Il23r can be selected using methods known in the art. Exemplary methods for detecting gene expression include, e.g., PCR-based methods, chip-based methods, hybridization based methods, and protein detection by antibodies methods.

The sequences of the mRNAs for IL10, Pcsk1, Areg, Pcyt1a, Frmd5, Ccr1, Ccr3, Lyn, Arnt2, Pparg, Ctsh, Havcr2(TIM3), Gpr55, Il23r, Itgae, Ccr6, Dgat2, Rorc, CD74, Il1r2, Il1r11 (ST2), CD200r1, Npnt, and Trf 1 are available in public databases, e.g., as follows: Homo sapiens proprotein convertase subtilisin/kexin type 1 (Pcsk1) e.g., GI:295424141 or GI295789016; Homo sapiens CCR1, e.g., GI: 53759124; Homo sapiens alpharegulin (Areg), e.g., GI: 22035683; Homo sapiens phosphate cytidylyltransferase 1, choline, alpha (PCYT1A), e.g., GI 31543384; Homo sapiens Hepatitis A Virus cellular receptor or T cell immunoglobulin mucin 3 (Havcr1 or TIM3), e.g., GI: 49574533; Homo sapiens solute carrier family 15, member 3 (SLC15A3), e.g., GI: 226371631; CCR3, e.g., GI: 257743050; IL10, e.g., GI:24430216; Homo sapiens nephronectin (NPNT), e.g., GI: 296011072, GI: 296011070, GI: 296011068, GI: 296011066, or GI: 296011065; Homo sapiens cathepsin H (CTSH) e.g., GI 148536857; Homo sapiens aryl-hydrocarbon receptor nuclear translocator 2 (ARNT2), e.g., GI: 68303554; Homo sapiens IL23R, e.g., GI: 24430211; Homo sapiens phosphate cytidylyltransferase 1, choline, alpha (PCYT1A), e.g., GI: 31543384; Homo sapiens FERM domain containing 5 (FRMD5), e.g. GI: 94721307; Homo sapiens FERM domain containing 5 (FRMD5), e.g., GI: 94721307; Homo sapiens peroxisome proliferator-activated receptor gamma (PPARG), e.g., GI: 226061859; Homo sapiens G protein-coupled receptor 55 (GPR55), e.g., GI: 115345344; Homo sapiens integrin, alpha E (antigen CD103, human mucosal lymphocyte antigen 1; alpha polypeptide) (ITGAE), e.g., GI: 148728187; Homo sapiens chemokine (C—C motif) receptor 6 (CCR6), e.g., GI: 150417991; Homo sapiens diacylglycerol O-acyltransferase 2 (DGAT2), e.g., GI: 189458880; Homo sapiens RAR-related orphan receptor C(RORC), e.g. GI: 48255917; Homo sapiens RAR-related orphan receptor C(RORC), e.g. GI: 48255916; Homo sapiens CD74 molecule, major histocompatibility complex, class II invariant chain (CD74), e.g., GI: 68448543; Homo sapiens interleukin 1 receptor-like 2 (IL1RL2), e.g., GI: 28416901; Homo sapiens interleukin 1 receptor-like 1 (ST2), e.g., GI: 27894323 or 27894327; Homo sapiens CD200r1, e.g., GI: 41327722 or 68215526 or 68215643 or 41327723; Homo sapiens telomeric repeat binding factor (NIMA-interacting) 1 (TERF1), e.g., GI: 189409141; Homo sapiens nephronectin (NPNT), e.g., GI: 296011072 or 296011070 or 296011068 or 296011066 or 296011065.

It will be understood that, in addition to these exact sequences, sequences that are substantially identical to these sequences may be selected by one having ordinary skill in the art. As used herein, “substantially identical” refers to a nucleotide sequence that contains a sufficient or minimum number of identical or equivalent nucleotides to the reference sequence, such that homologous recombination can occur. For example, nucleotide sequences that are at least about 80% identical to the reference sequence are defined herein as substantially identical. In some embodiments, the nucleotide sequences are about 85%, 90%, 95%, 99% or 100% identical.

To determine the percent identity of two nucleic acid sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 80% (in some embodiments, about 85%, 90%, 95%, or 100%) of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package, using a Blossum 62 scoring matrix with a gap penalty of 12, a gap extend penalty of 4, and a frameshift gap penalty of 5.

### III. METHODS OF USE

In one embodiment, a population of cells comprising tissue-regenerative Treg cells is administered to a subject by an appropriate route. In one embodiment, such administration results in a therapeutic benefit to the subject. In one embodiment, a population of cells comprising tissue-regenerative Treg cells can be used to treat a disease or disorder. In another embodiment, a population of cells comprising tissue regenerative Treg cells can be used in the preparation of a medicament for modulation of wound healing. In one embodiment, the transplanted tissue regenerative Treg are syngeneic to the recipient. In another embodiment, the transplanted tissue-regenerative Treg cells are allogeneic to the recipient. In yet another embodiment, the transplanted tissue regenerative Treg cells are xenogeneic to the recipient. Accordingly, in various embodiments, the invention provides use of a composition of the invention comprising tissue-regenerative Treg cells for therapy or for treatment of a disease or disorder. The invention also provides use of a composition of the invention comprising tissue regenerative T reg cells for the manufacture of a medicament for modulating (e.g., promoting) wound healing.

In another embodiment, one or more agents regulating the differentiation of tissue-regenerative Treg cells can be administered to a subject. In one embodiment, such administration results in a therapeutic benefit to the subject. In one embodiment, an agent that promotes the differentiation of tissue regenerative Treg cells can be used to treat a disease or disorder. In another embodiment, an agent that promotes the differentiation of tissue regenerative Treg cells can be used in the preparation of a medicament for modulation of wound healing.

Exemplary such agents include peroxisome proliferator-activated receptor γ (PPARγ) agonists. Exemplary such agonists include Thiazolidinedione-like drugs or TZDs which act by binding to PPARγ. Exemplary such agents include Rosiglitazone (Avandia), Pioglitazone (Actos), and Troglitazone (Rezulin), Galida (tesaglitazar), and Aleglitazar. Other agents include MCC-555, a powerful antidiabetic agent, rivoglitazone, and ciglitazone.

In another embodiment, IL-2/anti-IL-2 complexes, or genetically engineered IL-2, can be used to expand the population of tissue regenerative Treg cells.

In another embodiment, one or more agents produced by tissue-regenerative Treg cells or a soluble derivative thereof, (e.g., a fusion protein) or an agent which binds to an agent produced by the tissue-regenerative Treg cells can be administered to a subject. In one embodiment, such administration results in a therapeutic benefit to the subject. In one embodiment, an agent which is produced by tissue regenerative Treg cells can be used to treat a disease or disorder. In another embodiment, an agent that is produced by tissue-regenerative Treg cells can be used in the preparation of a medicament for modulation of wound healing.

Exemplary such factors include one or more of: IL-10, Areg (amphiregulin), Havcr2 (TIM3) and Npnt (nephronectin) (soluble derivatives thereof, e.g., fusion proteins) or agents which bind thereto.

It will be understood that each of these compositions of the invention, e.g., populations of tissue-regenerative Treg cells, agents that regulate the differentiation of tissue-regenerative Treg cells and agents that are produced by tissue-regenerative Treg cells can be used in the methods described herein. It will also be understood that such compositions may be administered alone. In another embodiment, two such compositions may be administered to a subject (e.g., a composition comprising tissue regenerative Treg cells and at least one agent that promotes their growth and/or differentiation or at least one agent produced by such cells). In another embodiment, all three compositions may be administered.

In one embodiment, the subject is a mammal. In one embodiment, the subject is a human. In another embodiment, the subject is a domesticated animal.

In one embodiment, a composition of the invention can be administered to a subject having a wound, e.g., a wound to muscle tissue. In another embodiment, a composition of the invention can be administered to a subject having an injury, e.g., an injury to muscle tissue. In one embodiment, the injury is a sports injury. In another embodiment, a composition of the invention can be administered to an individual having undergone strenuous exercise. Accordingly, the invention also provides for use of a composition comprising tissue-regenerative Treg cells for the manufacture of a medicament for the treatment of an injury, such as an injury to muscle tissue or a sports injury.

In one embodiment, the wounded muscle tissue is skeletal muscle tissue. In another embodiment, the wounded muscle tissue is smooth muscle tissue. In yet another embodiment, the wounded muscle tissue is cardiac muscle tissue.

In one embodiment, a composition of the invention is administered in conjunction with transplantation of cells, e.g., muscle cells and/or cells capable of differentiating into muscle cells (e.g., muscle stem cells, and/or muscle progenitor cells).

In one embodiment, a composition of the invention is administered to a subject of advanced age.

In another embodiment, a composition of the invention is administered to a subject having muscle degeneration, wasting or atrophy. In one embodiment, the muscle wasting or atrophy is the result of an injury or paralysis. In another embodiment, the muscle wasting or atrophy is the result of an inherited condition. Accordingly, the invention also provides for use of a composition comprising tissue-regenerative Treg cells in the manufacture of a medicament for treatment of a disease or disorder characterized by muscle degeneration, muscle wasting or muscle atrophy.

In one embodiment, the subject has a disorder characterized by impaired wound healing. In one embodiment, the subject has diabetes. Use of a composition of the invention for the manufacture of a medicament for the treatment of a disorder characterized by impaired wound healing or for treatment of diabetes is also provided.

In one embodiment, administration of a composition of the invention can be, for example, parenteral (e.g., by subcutaneous, intrathecal, intraventricular, intramuscular, or intraperitoneal injection, bronchial injection or by intravenous drip); topical (e.g., transdermal, ophthalmic, or intranasal); or pulmonary (e.g., by inhalation or insufflation of powders or aerosols). Administration can be rapid (e.g., by injection) or can occur over a period of time (e.g., by slow infusion or administration of slow release formulations). In one embodiment, a composition of the invention is administered intravenously. In yet another embodiment, a composition of the invention is administered subcutaneously.

Systemic administration of a composition of the invention can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In another embodiment, a composition of the invention (e.g., tissue-regenerative Treg cells) may be administered to recipients by injection into an allograft or into a surgical field into which the allograft is implanted, or any combination thereof.

In one embodiment, a composition of the invention is administered directly to a wound. In another embodiment, a composition of the invention is administered directly to muscle, e.g., wounded muscle tissue.

In one embodiment, a composition of the invention is formulated for administration. In the case of cellular compositions, appropriate carriers or vehicles for administration (e.g., for pharmaceutical administration) of cells are compatible with cell viability and are known in the art. Such carriers may optionally include buffering agents or supplements. In the case of cellular compositions, such supplements may promote cell viability. In one embodiment, a composition is formulated with one or more additional agents, e.g., survival-enhancing factors or pharmaceutical agents. In one embodiment, cells are formulated with a liquid carrier that is compatible with survival of the cells.

In one embodiment, a composition of the invention is formulated with a pharmaceutically acceptable carrier. As used herein, “pharmaceutically acceptable carriers” includes saline, solvents, dispersion media, coatings, antibacterial and antifungal agents, isotonic and absorption-delaying agents, and the like, compatible with pharmaceutical administration.

Pharmaceutical compositions are typically formulated to be compatible with their intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are typically delivered in the form of an aerosol spray from a pressurized container or dispenser that contains a suitable propellant, e.g., a gas such as carbon dioxide, or a nebulizer. Such methods include those described in U.S. Pat. No. 6,468,798.

The therapeutic compounds can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

For administration of cells, the quantity of induced tissue-regenerative cells to be administered to a subject can be determined by one of ordinary skill in the art. In one embodiment, amounts of cells can range from about 10⁵ to about 10¹⁰ cells per dose. In exemplary embodiments, cells are administered in a quantity of about 10⁵, 10⁶, 10⁷, 10⁸, 10⁹, or 10¹⁰ cells per dose. In other exemplary embodiments, intermediate quantities of cells are employed, e.g., 5×10⁵, 5×10⁶, 5×10⁷, 5×10⁸, 5×10⁹, or 5×10¹⁰ cells. In some embodiments, subjects receive a single dose of cells. In other embodiments, subjects receive multiple doses. Multiple doses may be administered at the same time, or they may be spaced at intervals over a number of days. For example, after receiving a first dose, a subject may receive subsequent doses of cells at intervals of 1, 2, 3, 4, 5, 6, 7, 8, 9, 10, 11, 12, 13, 14, 21, 28, 30, 45, 60, or more days.

As will be apparent to one of skill in the art, the quantity of cells and the appropriate times for administration may vary from subject to subject depending on factors including the duration and severity of disease. To determine the appropriate dosage and time for administration, skilled artisans may employ conventional clinical and laboratory means for monitoring the outcome of administration, e.g., on progression of a disorder in the subject. Such means include known biochemical and immunological tests for monitoring and assessing, for example, muscle strength, muscle mass, wound healing, etc. In another embodiment, change in cellular composition of tissue, e.g., at the site of injury as measured using methods known in the art, e.g., a change in muscle infiltrate from that dominated by a pro-inflammatory phenotype (CD11b+Lytic high) to that dominated by an anti-inflammatory phenotype (CD11b+Ly6c low) can be observed.

Prophylactic administration of a composition of the invention can be initiated prior to the onset of disease or therapeutic administration can be initiated after a disorder is established or, e.g., after a wound has been received.

In one embodiment, administration of a composition of the invention is undertaken e.g., prior to receipt of a transplant. In exemplary embodiments, cells may be administered at one or more times including, but not limited to, 30, 14, 13, 12, 11, 10, 9, 8, 7, 6, 5, 4, 3, 2, 1, or 0 days prior to transplantation. In addition or alternatively, composition of the invention can be administered to a recipient following transplantation. In exemplary embodiments a composition of the invention is administered at one or more times including, but not limited to, 1, 2, 3, 4, 5, 6, 7, 8, 9, 10, 11, 12, 13, 14, 30, etc. days following transplantation. In one embodiment, such a transplant is a transplant of muscle cells, muscle cell progenitors, and/or muscle stem cells. In one embodiment, the transplanted cells are syngeneic to the recipient. In another embodiment, the transplanted cells are allogeneic to the recipient. In yet another embodiment, the transplanted cells are xenogeneic to the recipient.

Dosage, toxicity and therapeutic efficacy of therapeutic compositions as described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. In general, when the IL-2:anti-IL-2 monoclonal antibody (mAb) complex is administered, or genetically engineered IL-2 is administered, a preferred dosage will be sufficient to increase numbers of tissue-regenerative Tregs without increasing the number of effector T cells.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. Such information can be used to more accurately determine useful doses in humans. Levels in plasma may be measured, for example, by high performance liquid chromatography.

A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

In some embodiments, administration of a composition of the invention can be accompanied by administration of one or more additional agents. For example tissue-regenerative Treg cells can be administered with one or more immunosuppressive agents. Exemplary immunosuppressive agents that can be used in combination with the induced compositions described herein include, but are not limited to, cytokines such as, for example, interleukin-10, and/or pharmaceutical agents such as, for example, corticosteroids, methotrexate, NSAIDs, fingolimod, natalizumab, alemtuzumab, anti-CD3, cyclosporine A and tacrolimus (FK506). In preferred embodiments, the use of a composition of the invention will allow for administration of lower doses of general immunosuppressants than the current standard of care, thereby reducing side effects.

In some embodiments, e.g., where the tissue-regenerative Tregs were not obtained from the recipient, the methods described herein can include the use of minimal hematoablative conditioning of the recipient. In some embodiments, minimal hematoablative conditioning can include the use, e.g., transitory use, of low doses of one or more chemotherapy agents, e.g., vincristine, actinomycin D, chlorambucil, vinblastine, procarbazine, prednisolone, cyclophosphamide, doxorubicin, vincristine, prednisolone, lomustine, and/or irradiating the thymus of the recipient mammal, e.g., human, with a low dose of radiation, e.g., less than a lethal dose of radiation plus chemotherapy agents. Lethal doses of conditioning include the administration of 14 Gy of irradiation plus cytarabine, cyclophosphamide, and methylprednisolone (Guinin et al, New Engl. J. Med., 340:1704-1714, 1999).

To prevent the development of graft-versus-host disease, additional treatment with a short course of methotrexate and cyclosporine starting on the day before transplantation using a bolus of 1.5 mg/kg over a period of 2-3 hours every 12 hours. This protocol should allow the reduction of irradiation conditioning to about 10 Gy or less, e.g., in some embodiments, about 5 Gy, about 2 Gy, about 1.5 Gy, about 1 Gy, about 0.5 Gy, about 0.25 Gy and the elimination of additional cytoreduction agents such as cytarabine, cyclophosphamide, and methylprednisolone treatments. Minimal hematoablative conditioning is typically achieved by administering chemical or radiation therapy at a level that will not destroy the recipient's immune function, and is similar to, or lower than, levels used for conventional cancer treatments, e.g., conventional chemotherapy.

In one embodiment, the invention pertains to a method of promoting wound healing comprising contacting muscle cells with a composition of the invention.

In one embodiment, the method is performed in vivo by administering a composition of the invention to a subject. In one embodiment, the administration results in a desired effect in the subject, e.g., promotion of wound healing and/or differentiation of increased muscle tissue.

In another embodiment, a composition of the invention can be used to promote the differentiation of muscle cells ex vivo. For example, in one embodiment a population comprising tissue regenerative Tregs can be contacted with muscle cells or cells capable of differentiating into muscle cells (e.g., muscle stem cells or progenitor cells). In one embodiment, the cells capable of differentiating into muscle cells are further contacted with macrophages or at least one agent produced by macrophages. In another embodiment, the cells capable of developing into muscle cells are further contacted by at least one agent selected from the group consisting of: at least one cytokine, muscle cell extract, and at least one myokine.

### IV. METHODS OF IDENTIFYING ADDITIONAL WOUND HEALING AGENTS

In one embodiment, the invention pertains to the identification of agents that directly or indirectly enhance wound healing. As set forth herein, agents that enhance the proliferation and/or differentiation of tissue-regenerative Tregs can be used to promote wound healing.

In one embodiment, a screening method of the invention employs a tissue-regenerative Treg cell, e.g., a tissue-regenerative Treg cell that has been isolated from muscle.

In one embodiment, tissue-regenerative Treg cells can be treated with a test agent and proliferation of the cell can be tested to determine whether the agent augments proliferation of the cells. Agents that do augment proliferation of these cells as compared with appropriate controls are potentially useful for producing induced tissue-regenerative Treg cells.

In another embodiment, a traditional Treg cell can be contacted with a test agent and the ability of the test agent to convert the phenotype of the Treg cell to that of a tissue-regenerative Treg cell can be tested. Agents that enhance the conversion of traditional Treg cells to that of tissue-regenerative Treg cells as compared with appropriate controls are potentially useful for producing induced tissue regenerative Treg cells. Such agents may also convert CD4⁺Foxp3⁻ T conventional cells to tissue-regenerative Treg cells.

A variety of test compounds can be evaluated using the screening assays described herein. The term “test compound” includes reagents or test agents that are employed in the assays of the invention and assayed for their ability to influence tissue regeneration. More than one compound, e.g., a plurality of compounds, can be tested at the same time for their ability to modulate tissue regeneration or gene expression in a screening assay. The term “screening assay” preferably refers to assays that test the ability of a plurality of compounds to influence the readout of choice rather than to tests that test the ability of one compound to influence a readout. Preferably, the subject assays identify compounds not previously known to have the effect that is being screened for. In one embodiment, high-throughput screening can be used to assay for the activity of a compound.

In certain embodiments, the compounds to be tested can be derived from libraries (i.e., are members of a library of compounds). While the use of libraries of peptides is well established in the art, new techniques have been developed that have allowed the production of mixtures of other compounds, such as benzodiazepines (Bunin et al. (1992). J. Am. Chem. Soc. 114:10987; DeWitt et al. (1993). Proc. Natl. Acad. Sci. USA 90:6909), peptoids (Zuckermann. (1994). J. Med. Chem. 37:2678), oligocarbamates (Cho et al. (1993). Science. 261:1303-), and hydantoins (DeWitt et al. supra).

Exemplary methods used to generate molecular diversity are well known in the art and many reviews have been published, e.g., Shreiber, S. (2009) Nature 457, 153-154; Barry, C. E. I. (2003), 2, 137-150.; Braeckmans, K. et al. (2003) Encoded microcarrier beads signal the way to better combinatorial libraries and biological assays. Mod. Drug Dis., 6, 28-30, 32; Charmot, D. (2003) Actualite Chimique, 11-16; Edwards, P. J. (2003), 6, 11-27; Fassina, G., & Miertus, S. (2003) Chimica Oggi, 21, 28-31; Hermkens, P. H. H., & Muller, G. (2003). Ernst Schering Research Foundation Workshop, 42, 201-220.; Hisamoto, H., Kikutani, Y., & Kitamori, T. (2003) Microchip-based organic synthesis. Shokubai, 45, 252-256; Hughes, D. (2003). Nature Reviews Genetics, 4, 432-441; Jensen, K. J., & Nielsen, J. (2003) Bioorganic and combinatorial chemistry. Part 1. Dansk Kemi, 84, 21-24; Kobayashi, N., & Okamoto, Y. (2003) Farumashia, 39, 769-773.; Lam, K. S., Liu, R., Miyamoto, S., Lehman, A. L., & Tuscano, J. M. (2003). Account. Chem. Res., 36, 370-377; Langer, T., & Krovat, E. M. (2003), 6, 370-376; Liu, R., Enstrom, A. M., & Lam, K. S. (2003). Experimental Hematology (New York, N.Y., United States), 31, 11-30.; Mario Geysen, H., Schoenen, F., Wagner, D., & Wagner, R. (2003) Nature Reviews Drug Discovery, 2, 222-230; Nefzi, A., Ostresh, J. M., & Houghten, R. A. (2003). EXS, 93, 109-123.; New, D. C., Miller-Martini, D. M., & Wong, Y. H. (2003). Phytotherapy Research, 17, 439-448. Pinilla, C., Appel, J. R., Borras, E., & Houghten, R. A. (2003) Nature Medicine (New York, N.Y., United States), 9, 118-122; Schwardt, O., Kolb, H., & Ernst, B. (2003) Current Topics in Medicinal Chemistry (Hilversum, Netherlands), 3, 1-9.; Sehgal, A. (2003). Curr. Med. Chem., 10, 749-755. The contents of these reviews are incorporated by reference herein.

The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). Other exemplary methods for the synthesis of molecular libraries can be found in the art, for example in: Erb et al. (1994). Proc. Natl. Acad. Sci. USA 91:11422-; Horwell et al. (1996) Immunopharmacology 33:68-; and in Gallop et al. (1994); J. Med. Chem. 37:1233-.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310). In still another embodiment, the combinatorial polypeptides are produced from a cDNA library. In one embodiment, cDNA molecules for testing can be expressed in viral libraries, e.g., be retro-, lenti-, or adenoviral libraries. In another embodiment, RNAi libraries developed using methods known in the art can be screened.

Exemplary compounds that can be screened for activity include, but are not limited to, peptides, nucleic acids, carbohydrates, small organic molecules, and natural product extract libraries.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L- configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries); 5) enzymes (e.g., endoribonucleases, hydrolases, nucleases, proteases, synthatases, isomerases, polymerases, kinases, phosphatases, oxido-reductases and ATPases), or RNAi molecules.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Other examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In another embodiment, the effect of the compound of interest on the cells, is compared with an appropriate control (such as untreated cells or cells treated with a control compound, or carrier, that does not modulate the biological response).

In another embodiment, a test compound is identified that directly or indirectly modulates tissue regeneration, e.g., by one of the variety of methods described hereinbefore. The selected test compound (or “compound of interest”) can then be further evaluated in a secondary screening assay.

Compounds identified in the subject screening assays can be used in methods of modulating induction of tissue regenerative Treg cells and may also be appropriate for administration to subjects to enhance wound healing in vivo. It will be understood that it may be desirable to formulate such compound(s) as pharmaceutical compositions (described supra) prior to contacting them with cells.

In one embodiment, tissue regenerative Tregs can be used to identify factors produced by them (proteins, lipids, small molecules) that may act on muscle cells. For example, preparations of Treg extract, or Treg conditioned media can be contacted with muscle cells or muscle stem cells to determine the effect of these agents on muscle cells or stem cells.

### EXAMPLES

The invention is further described in the following examples, which do not limit the scope of the invention described in the claims.

### Example 1

### Increase in the Treg Population is Seen at the Site of Muscle Injury as Inflammation is Waning and Regeneration is Beginning

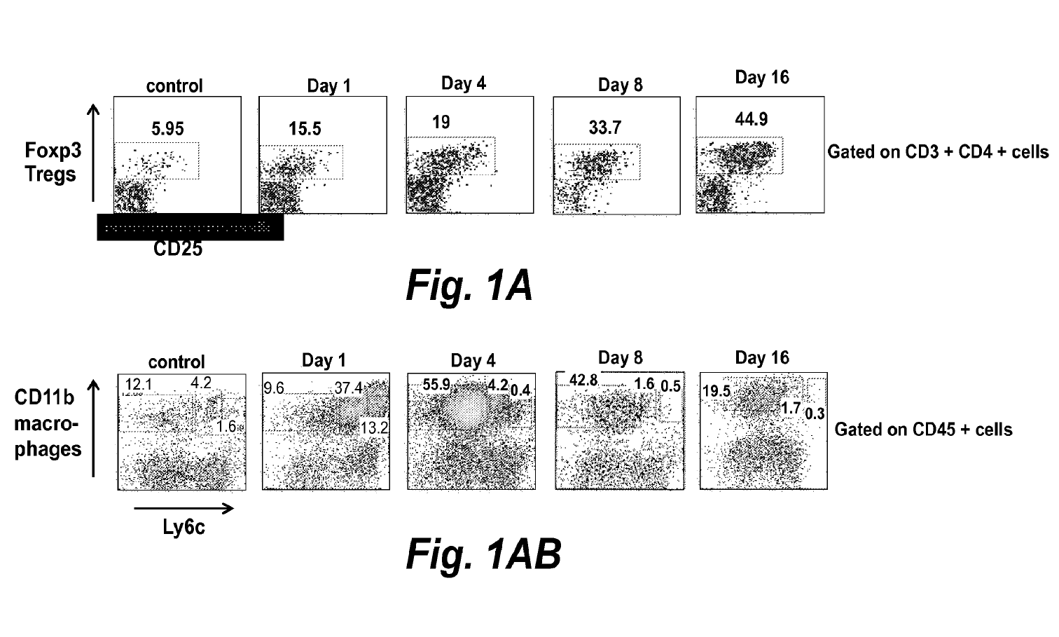

8-wk-old C57BL/6 mice were injured i.m. with cardiotoxin (30 μg/ml), and the muscle infiltrate was analyzed after 1, 4, 8 and 16 days by flow cytometry. Each sample was stained for analysis of Tregs (FIG. 1A) and myeloid cells (FIG. 1B). After the induction of muscle injury, the frequency of CD4⁺ Tregs (Foxp3⁺CD25⁺) gradually increases in the wounded muscle, leading to an accumulation of Tregs at the site of injury. In parallel, the inflammatory myeloid population that initially invades the muscle (CD11b⁺Ly6c high) shifts to an anti-inflammatory phenotype (CD11b⁺Ly6c low) that promotes tissue regeneration.

### Example 2

### Ablating Tregs Inhibits Muscle Regeneration after Wounding

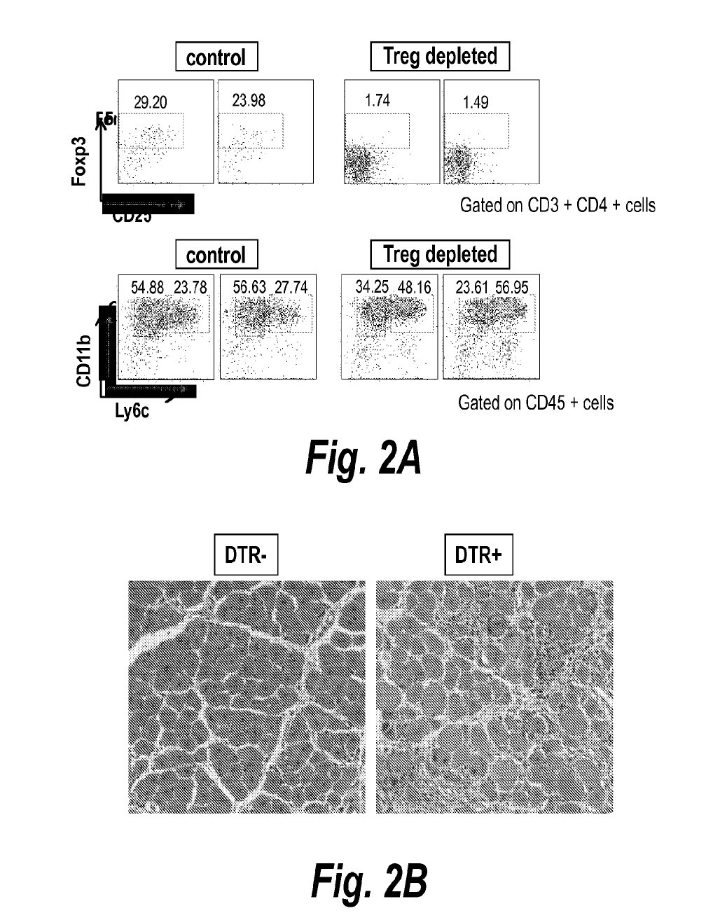

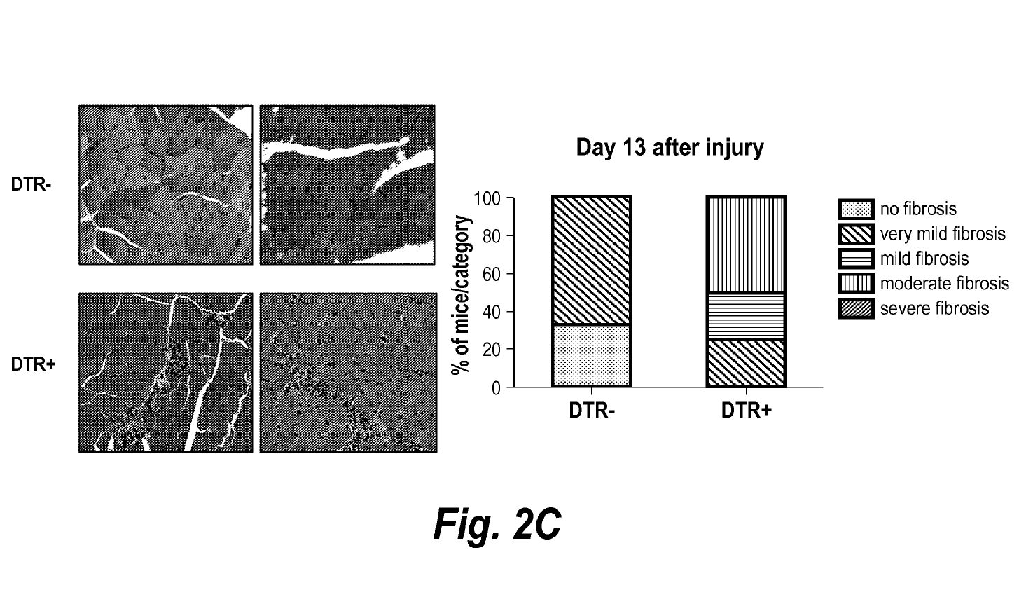

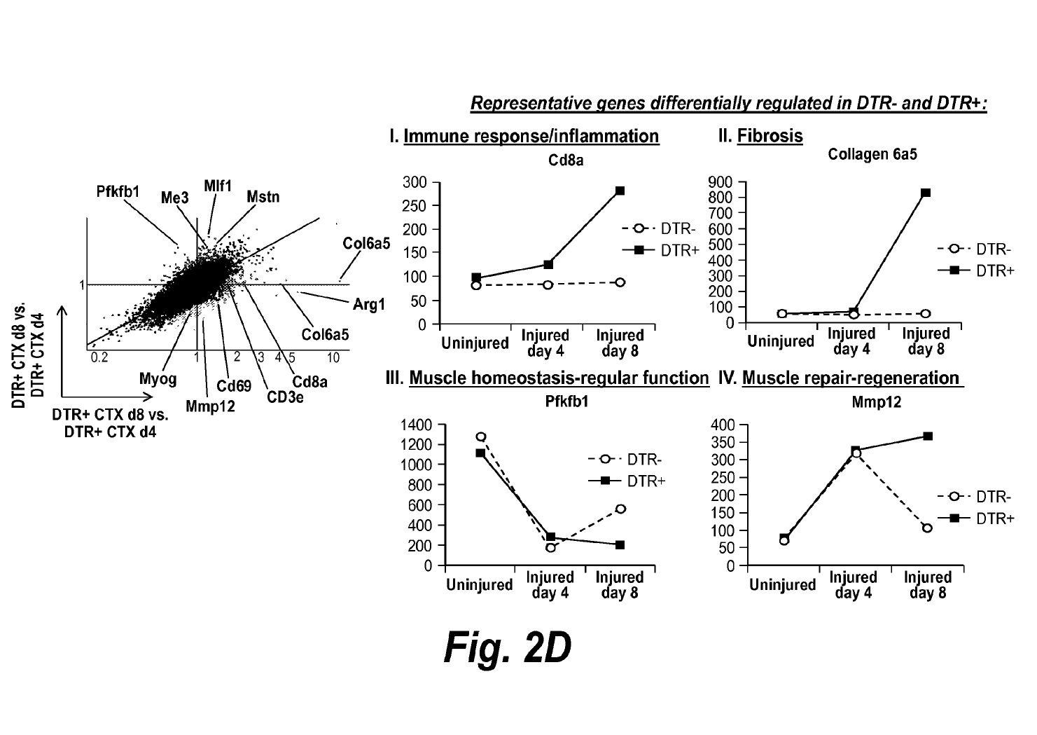

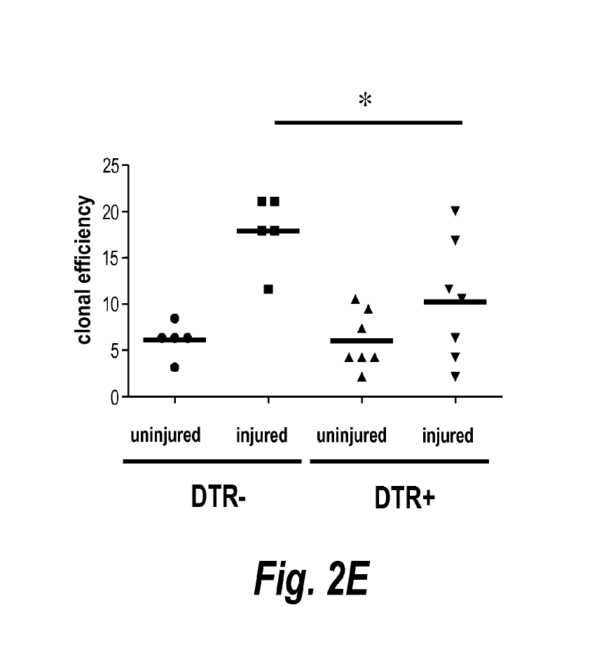

The Foxp3-DTR mouse model was used to achieve specific and temporal depletion of Tregs during the time of injury. 8-wk-old C57BL/6 Foxp3-DTR⁺ mice and their Foxp3-DTR⁻ littermates were treated with diptheria toxin (DT) to specifically deplete Tregs and then injured i.m. with cardiotoxin (30 μg/ml). After 8, days the muscle infiltrate was analyzed by flow cytometry (FIG. 2A) and muscle regeneration was analyzed by hematoxylin and eosin staining (FIG. 2B). When injury and repair occur in the absence of Tregs, the initial inflammation is not resolved, as shown by the accumulation of CD11b⁺Ly6c high monocytes (FIG. 2A) and persistence of a mononuclear infiltrate (FIG. 2B, right panel). In addition, in the absence of Tregs, muscle regeneration is impaired, as indicated by the reduced numbers of centrally nucleated (newly regenerated) myofibers in the injured area (FIG. 2B).

After 13 days, collagen accumulation was analyzed by Gomori's OneStep Trichrome staining, which is an art-accepted measure of fibrosis. The impairment in muscle repair after injury in the absence of Tregs is also measured as increased fibrosis levels (FIG. 2C).

After 4 or 8 days, muscle tissue was collected for microarray analysis. The impairment in muscle repair after injury in the absence of Tregs is also evidenced by alterations in the transcriptional profile of muscle regulation (FIG. 2D). Genes in the indicated clusters were differentially expressed in the absence of Tregs, indicating ineffective muscle repair.

After 4 days, skeletal muscle progenitors were single cell-sorted and cultured for 5 days to evaluate their colony formation efficiency. The impairment in muscle repair after injury in the absence of Tregs also is evidenced by the reduced clonal efficiency of the skeletal muscle progenitors (FIG. 2E).

### Example 3

### Treg Cells Isolated from Muscle are Unique

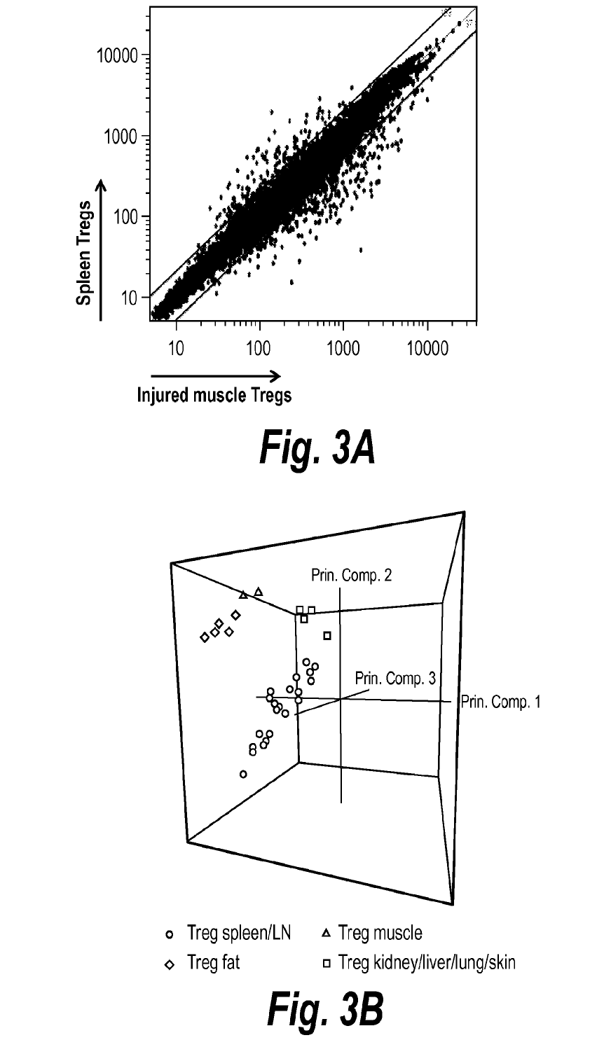

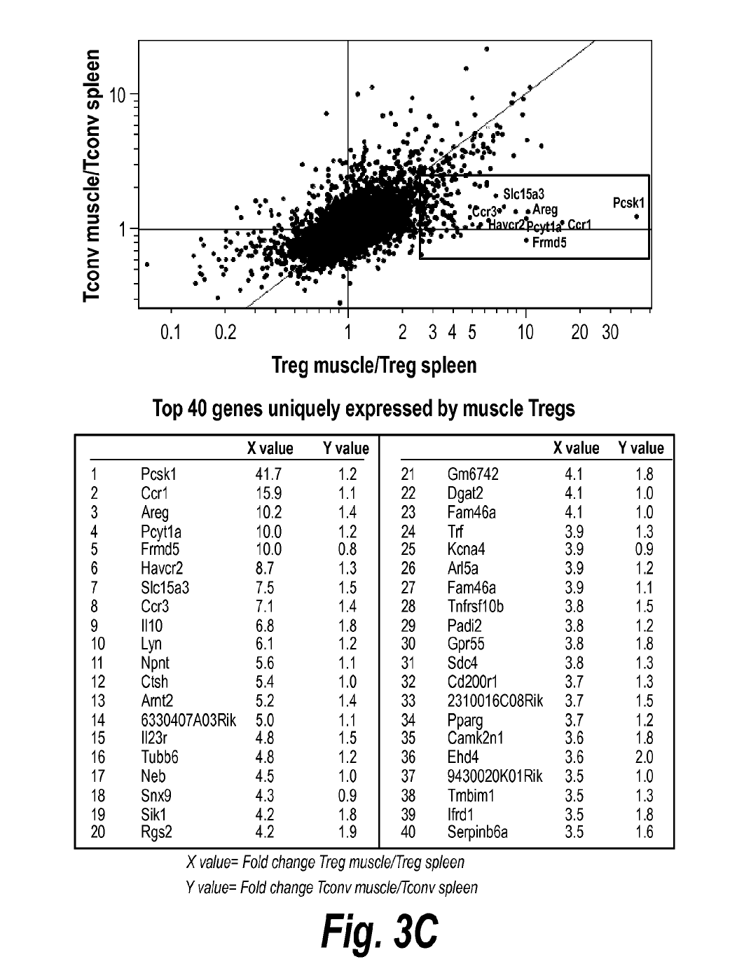

A comparative gene-expression analysis of Tregs from spleen and from injured skeletal muscle shows that more than 500 genes are differentially expressed (up- or down-regulated) between these two Treg populations (FIG. 3A). According to Principal Component Analysis (PCA) the muscle Tregs, are clearly different from Tregs isolated from all lymphoid tissues, and most closely resemble Tregs resident in fat (FIG. 3B). The anti-inflammatory molecules IL-10 and Havcr2 (TIM3) are two of the interesting genes uniquely expressed by muscle Tregs vis-à-vis other Tregs as well as T conventional cells from muscle or other tissues (FIG. 3C). Foxp3-IRES-GFP reporter mice were injured i.m. with cardiotoxin. After 2 weeks, muscle and spleen Treg cells were sorted and RNA was extracted to perform gene expression analysis by microarray, using the 1.0ST platform from Affymetrix. Tconv=T conventional cell (CD4⁺Foxp3⁻)

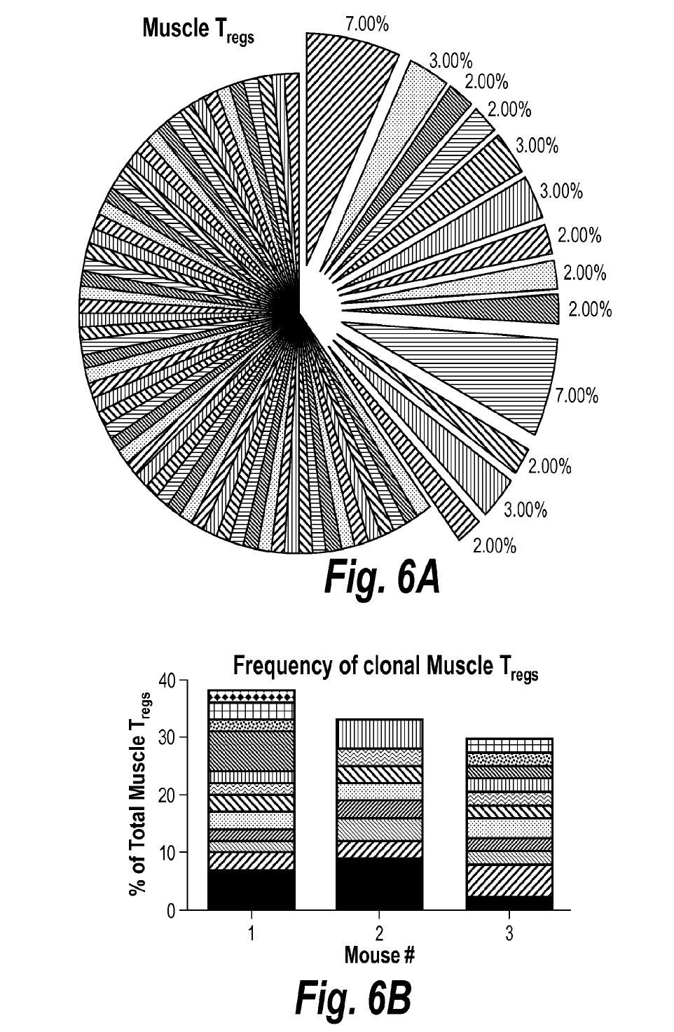

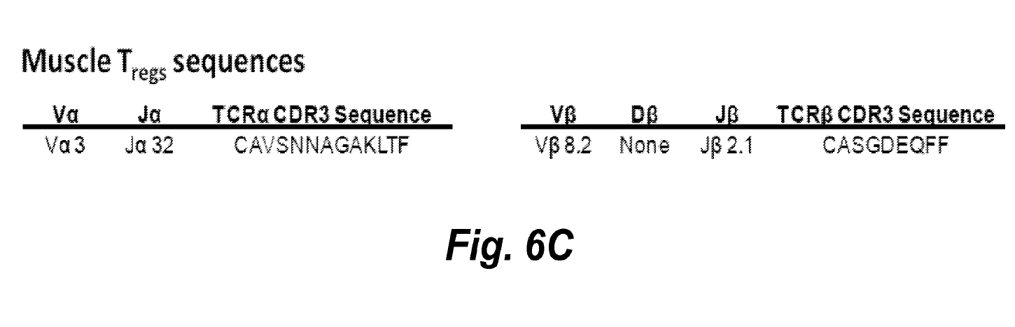

In additional experiments demonstrating that the Treg cell population isolated from muscle is unique, 8 week old C57BL/6-Foxp3-IRES-GFP mice were injured intramuscularly (i.m.) with cardiotoxin (30 μg/ml) and after 4 days Tregs from the muscle and spleen were single cell-sorted by flow cytometry. After PCR amplification, the CDR3 region of the TCRa and TCRb chains were sequenced and analyzed using IMGT/V-QUEST. The results show that a substantial portion of the Tregs isolated from injured muscle are clonally expanded, as shown in the example in FIG. 6A and in the summary in FIG. 6B. Between 30 and 40% of the TCR sequences of muscle Tregs belong to an expanded clone, while no identical sequences can be found in Tregs from the spleen. Interestingly, certain clones bearing the same TCRa and TCRb sequences can be found in individual mice in independent experiments (FIG. 6C). These data suggest that the Tregs are responding to a particular antigen in the muscle.

### Example 4

### An Expanded Population of Muscle Tregs is Found in Murine Models of Muscular Dystrophy

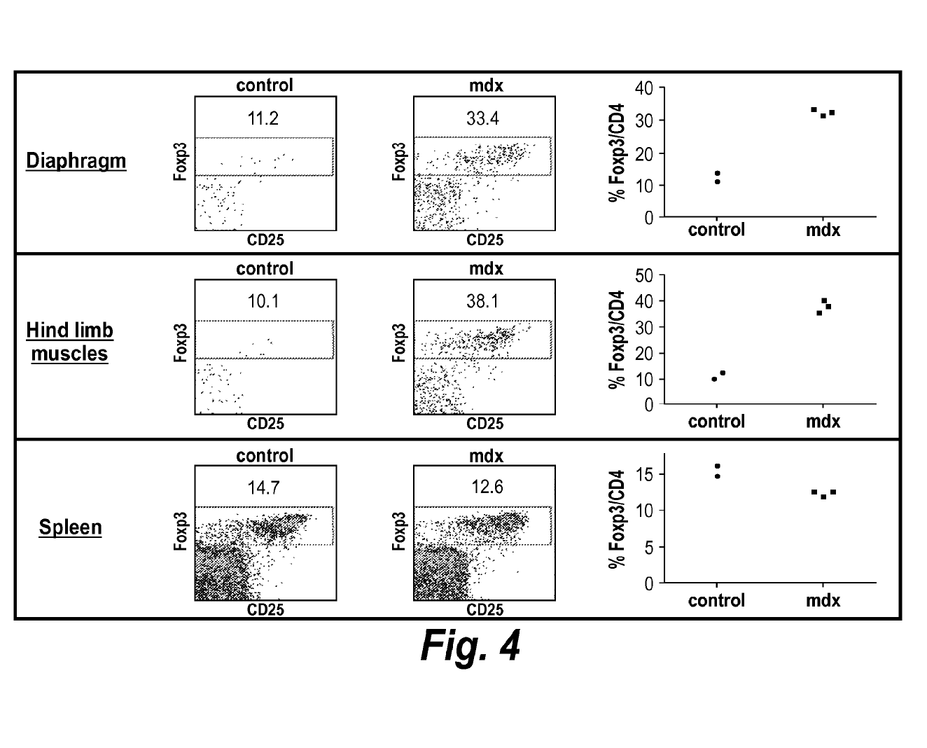

The immune cell infiltrate in the affected muscles of dystrophin-deficient (mdx) mice is enriched in Tregs. Thus, the increase in Treg frequency after muscle injury is not specific for acute, exogenously-induced injuries, but also occurs upon muscle damage in muscular dystrophies with genetic etiology (similar results were obtained with dysferlin-deficient mice). Would augmenting this population improve disease outcome? The infiltrate of diaphragms and hind limb muscles from 4 wk-old mdx (Murine X-linked muscular dystrophy) or control (C57BL/B10) mice were analyzed by flow cytometry (FIG. 4).

### Example 5

### A Reduced Population of Muscle Tregs is Found in Aged Mice

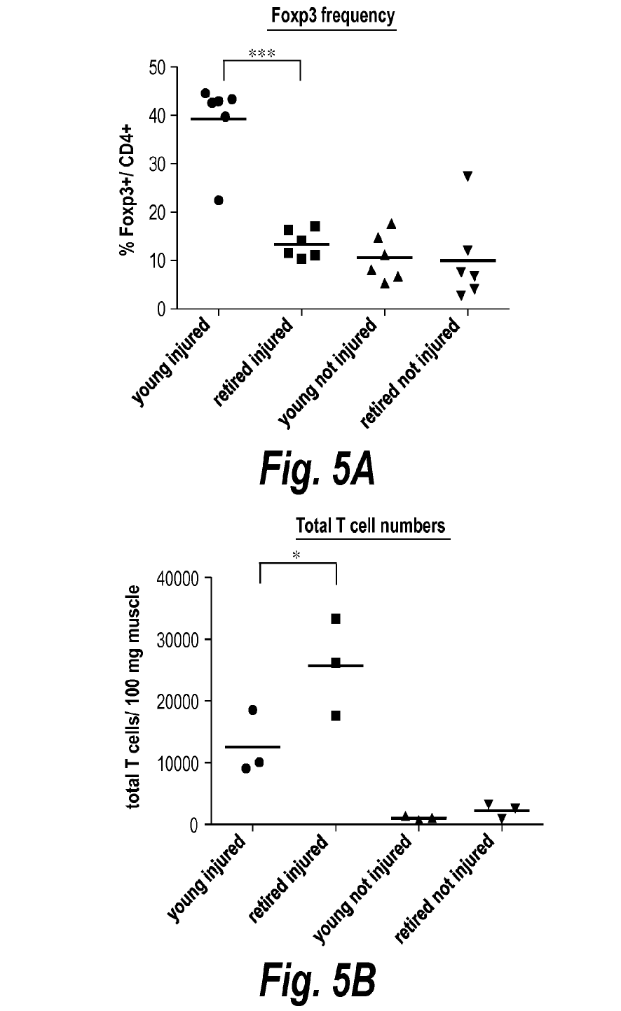

Young (8-wk-old) and retired (30-wk-old) C57BL/6 mice were injured i.m. with cardiotoxin (30 μg/ml) and the muscle infiltrate was analyzed after 8 days by flow cytometry. The increase in muscle Treg frequency after cardiotoxin injury is not observed in 30-wk-old mice (FIG. 5A). The low Treg frequency in older mice correlates with an increased accumulation of total T cells in the injured muscle (FIG. 5B). It has been previously reported that the regenerative capacity of muscle decreases with age, and this decreased regeneration is associated with a heightened or prolonged inflammatory response. Our results show that there are also changes in the composition of the immune infiltrate, which could be directly influencing the muscle repair process in old mice.

### Example 6

### Modulation of Treg Frequency Affects Muscle Damage in Dystrophic Mice

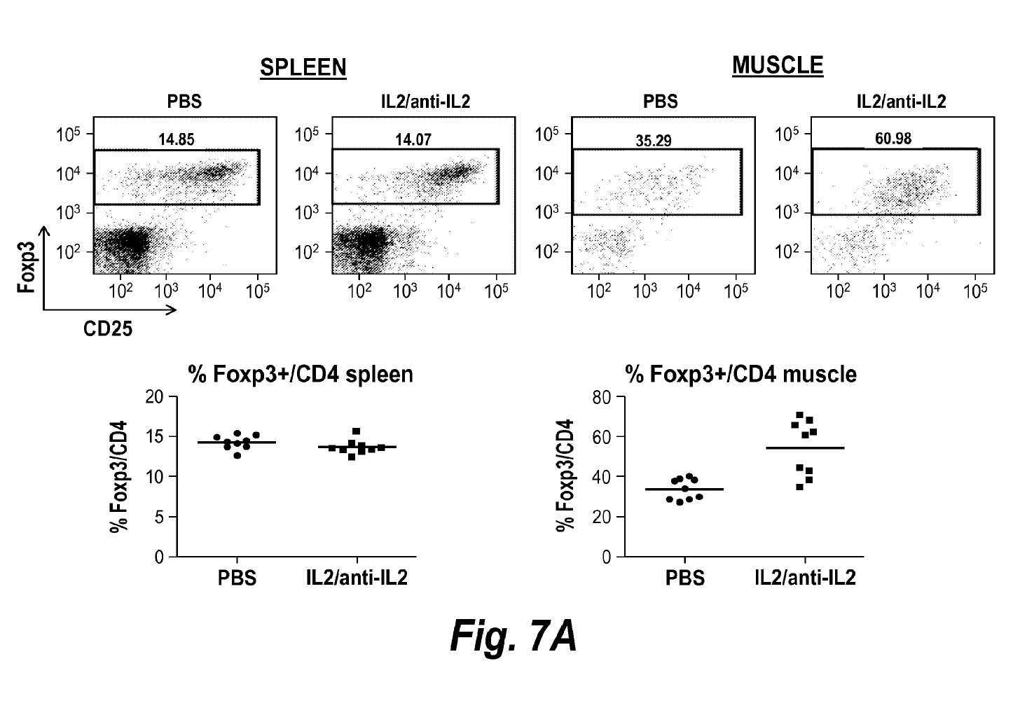

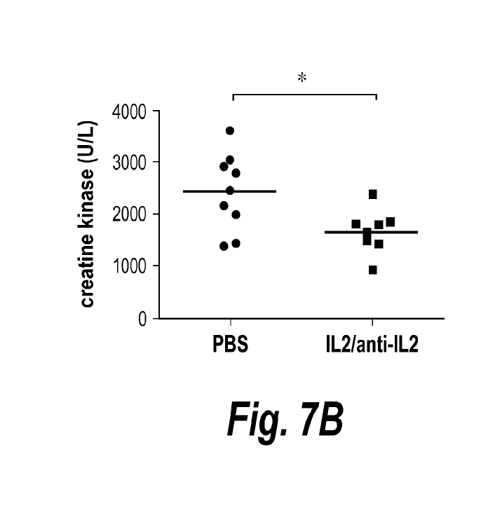

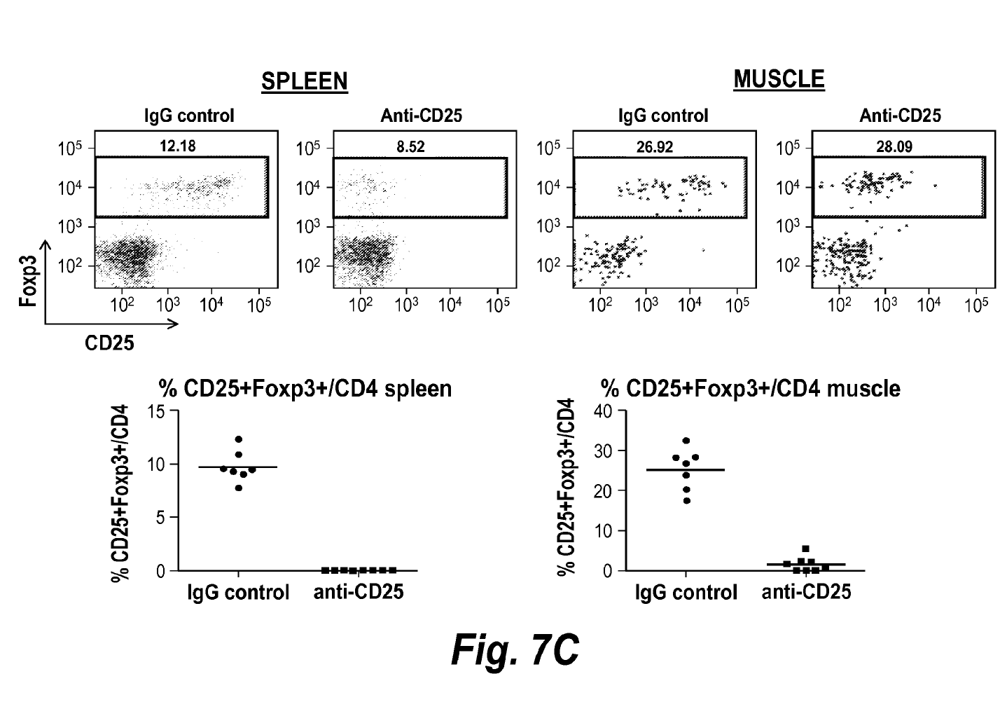

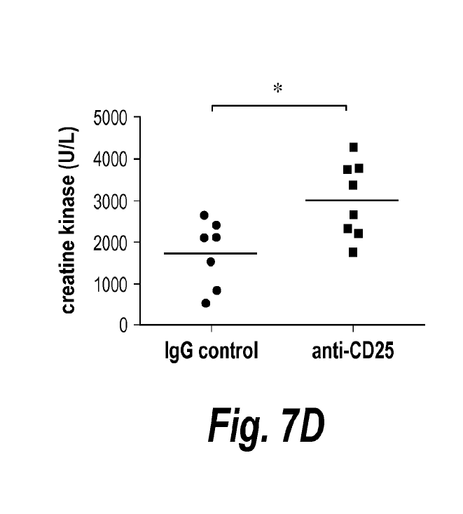

Seventeen day old Mdx male mice were treated with IL2/anti-IL2 complexes intraperitoneally (i.p.) for 6 consecutive days. Ten days after the last injection, the muscle infiltrate was analyzed by flow cytometry and the serum creatine kinase levels were assessed with the Creatine kinase-SL kit (Genzyme). As shown in FIG. 7A, the frequency of muscle Treg in dystrophic Mdx mice is augmented by treatment with IL2/anti-IL2 complexes. As shown in FIG. 7B, this increase in Treg correlates with a significant reduction in the levels of serum creatine kinase, a marker of muscle damage.

In a second set of experiments, Mdx mice were treated with anti-CD25 antibody (clone PC61) at days 17 and 20 of age. Seven days after the last injection, the muscle infiltrate was analyzed by flow cytometry and the serum creatine kinase levels were assessed with the Creatine kinase-SL kit (Genzyme). As shown in FIG. 7C, treatment with anti-CD25 (anti-IL2 receptor a) antibody (which is widely used to characterize Treg function in vivo) decreases the frequency of Tregs in the spleen but not in the muscle, although it affects CD25 expression in both tissues (FIG. 7C) and this correlates with increased muscle damage, as measured by increased levels of serum creatine kinase (FIG. 7D). Thus, modulation of Treg frequency can be considered a potential therapy to ameliorate muscle damage.

1. A composition of Foxp3+CD4+ regulatory T (Treg) cells isolated from muscle, which Treg cells exhibit tissue-regenerative properties, wherein the Treg cells are characterized by transcription of IL10, Pcsk1, Areg, Pcyt1a, Frmd5, Ccr1, Ccr3, Lyn, Arnt2, Pparg, Ctsh, Havcr2(TIM3), Gpr55, Il23r, Itgae, Ccr6, Dgat2, Rorc, CD74, Il1r2, Il1r11 (ST2), CD200r1 and Trf at levels higher than splenic, lymph node Treg cells, or conventional T cells.

2. (canceled)

3. A method of promoting wound healing in a subject in need thereof, comprising administering the composition of claim 1 to the subject.

4. The method of claim 3, wherein the composition is administered via a route selected from the group consisting of:
(a) systemically;
(b) directly to muscle tissue; and
(c) directly to a wound.

5-6. (canceled)

7. The method of claim 3, wherein the composition is administered to a subject having an injury to muscle tissue.

8. The method of claim 3, wherein the composition is administered to the subject at the time of injury or is administered to the subject several days after the injury.

9. (canceled)

10. The method of claim 3, wherein the subject is selected from the group consisting of:
(a) a subject having a degenerative muscle condition;
(b) a subject of advanced age; and
(c) a subject having diabetes.

11-12. (canceled)

13. The method of claim 3, further comprising administering an anti-inflammatory agent.

14. A method of promoting wound healing comprising contacting muscle cells with the composition of any of claim 1.

15. The method of claim 14, wherein the step of contacting occurs in vivo.

16. A method of producing a population of cells enriched for tissue regenerative Treg cells, the method comprising obtaining a starting population of cells comprising FoxP3+ CD4+ Treg cells and selecting or inducing cells from the starting population that express IL10, Pcsk1, Areg, Pcyt1a, Frmd5, Ccr1, Ccr3, Lyn, Arnt2, Pparg, Ctsh, Havcr2(TIM3), Gpr55, Il23r, Itgae, Ccr6, Dgat2, Rorc, CD74, Il1r2, Il1r11 (ST2), CD200r1 and Trf at levels higher than the bulk populations of splenic or lymph node circulating Treg cells and conventional T cells for all sites, to thereby produce a population of cells enriched for tissue regenerative Treg cells.

17. The method of claim 16, further comprising culturing the cells ex vivo in order to expand them.

18. The method of claim 17, wherein the cells are cultured in the presence of at least one agent selected from the group consisting of: at least one cytokine, muscle cell extract, and at least one myokine.

19. The method of claim 16, wherein the starting population of cells comprises cells derived from muscle.

20. A composition produced by the method of claim 16.

21. The method of claim 16, further comprising administering the cells to a subject.

22. The method of claim 16, further comprising contacting the population of cells enriched for tissue-regenerative Tregs with muscle cells or muscle cell progenitors.

23. The method of claim 22, wherein the step of contacting occurs in vivo.

24. A method of promoting wound healing comprising contacting muscle cells of a subject in need of wound healing with at least one agent that promotes the development of Treg cells, wherein the at least one agent is selected from the group consisting of: anti-CD3, at least one PPARγ agonist, at least one thiazolidinedione-like drug, an IL-2/anti-IL-2 complex, and pioglitazone.

25-26. (canceled)

27. A method of promoting wound healing comprising contacting muscle cells of a subject in need of wound healing with an agent derived from tissue regenerative Treg cells.

28. The method of claim 26, wherein the agent is selected from the group consisting of: IL-10, Areg (amphiregulin), Havcr2 (TIM3) and Npnt (nephronectin).

29. (canceled)

30. A method of promoting muscle cell differentiation ex vivo, comprising contacting cells capable of differentiating into muscle cells with a composition selected from the group consisting of:
(a) the composition of claim 1;
(b) macrophages;
(c) at least one agent produced by macrophages;
(d) at least one agent selected from the group consisting of: at least one cytokine, muscle cell extract, and at least one myokine; and
(e) one or more biological products selected from the group consisting of: proteins, RNAs, lipids, and other cellular molecules.

31-33. (canceled)

34. A pharmaceutical composition comprising the composition of claim 1 or 20.

35. A method for promoting wound healing and/or for the treatment of a degenerative muscle condition comprising administering the pharmaceutical composition of claim 34 to a subject in need thereof.

36. (canceled)
